(19) United States
(12) Patent Application Publication
(10) Pub. No.: US 2021/0283234 A1
(43) Pub. Date: Sep. 16, 2021

(54) ENGINEERED SALMONELLA SEROVAR TYPHIMURIUM STRAINS, COMPOSITIONS THEREOF, AND METHODS OF USE

(71) Applicant: North Carolina State University, Raleigh, NC (US)

(72) Inventors: HOSNI M. HASSAN, Raleigh, NC (US); STEPHEN BRYAN TROXELL, Raleigh, NC (US)

(21) Appl. No.: 16/321,795

(22) PCT Filed: Jul. 28, 2017
(86) PCT No.: PCT/US2017/044336

§ 371 Date: Jan. 29, 2019

(51) Int. Cl. A61K 39/112; A61P 31/04; C12N 15/10

(52) U.S. Cl. CPC A61K 39/0275; A61K 2039/55588; C12N 15/102; A61P 31/04

(57) ABSTRACT

Provided herein are engineered Salmonella enterica serovar Typhimurium strains and compositions, including vaccines, thereof. Also provided herein are methods of treating and/or preventing infection by at least Salmonella enterica serovar Typhimurium in a subject in need thereof by administering a vaccine provided herein.

### CROSS-REFERENCE TO RELATED APPLICATIONS

This application claims the benefit of and priority to co-pending U.S. Provisional Patent Application No. 62/368,507, filed on Jul. 29, 2016, entitled “ENGINEERED SALMONELLA SEROVAR TYPHIMURIUM STRAINS, COMPOSITIONS THEREOF, AND METHODS OF USE,” the contents of which is incorporated by reference herein in its entirety.

### STATEMENT REGARDING FEDERALLY SPONSORED RESEARCH OR DEVELOPMENT

This invention was made with government support under grant number 2012-68003-19621 awarded by the United States Department of Agriculture's National Institute of Food and Agriculture (USDA NIFA). The government has certain rights to this invention.

### BACKGROUND

Foodborne infections caused by Salmonella enterica serovars are a significant problem worldwide. The non-Typhoid Salmonella (NTS) are problematic because these serovars may be able to infect multiple animal hosts. As such there exists an unmet need for improved preventatives and treatments for NTS.

### SEQUENCE LISTING

This application contains a sequence listing filed in electronic form as an ASCII.txt file entitled 221404-2110_ST25.txt, created on Jul. 28, 2017. The content of the sequence listing is incorporated herein in its entirety.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a schematic of an embodiment of a vaccination protocol used to vaccinate mice with a modified S. Typhimurium. The following symbols represent the different treatments at the specified time points: V=Vaccination; B=Boosting; C1=1^{st} Challenge; and C2=2^{nd} Challenge. In some experiments described herein, C57BL/6 or BALBc female mice (6-8 weeks of age) underwent the vaccination/challenge protocol outlined in FIG. 1. In each experiment, two groups of mice (vaccinated and control) were subjected to the protocol in FIG. 1. Each mouse in the vaccinated group received about 100 μl containing about 10⁷ CFU of strain NC983, while each mouse in the Naïve control group received an equal volume of a PBS solution. In a second experiment, venous blood from the tail was collected 1-day prior to V, B, and C1—as indicated by the open arrows [symbol] for assaying anti-Salmonella IgG response.

FIG. 2 shows a graph demonstrating percent survival of C57BL/6 mice subjected to the vaccination protocol shown in FIG. 1. Two groups of mice (control group, n=3; and vaccine group, n=6) were subjected to the vaccination protocol outlined in FIG. 1. Mice in the vaccine group received oral doses of NC983 equivalent to about 6×10⁷ CFU/mouse and about 5×10⁸ CFU/mouse for vaccination and boosting, respectively. Mice in the control group received PBS. Mice in both groups were challenged with S. Typhimurium strain (NC1189) at about 2×10⁵ CFU/mouse (100×LD₅₀); and surviving mice were challenged again with a higher dose of 4×10⁶ CFU/mouse (>1000×LD₅₀). Survival of mice was monitored over time and expressed as percent. Statistical comparison of survival curves using Log-ranked (Mantel-Cox) test showed a p-value of 0.0397.

FIG. 3 shows a graph demonstrating percent survival of BALB/c mice subjected to the vaccination protocol shown in FIG. 1. Two groups of mice (control group, n=5; and the vaccine group, n=6) were subjected to the vaccine protocol described in FIG. 1. At V and B, each mouse in the vaccinated group received 9×10⁶ CFU/mouse and 1×10⁸ CFU/mouse of NC983, respectively; while the control mice received PBS. At C1 and C2, mice were challenged with the virulent strain NC1040 at 6×10⁴ and 1.2×10⁶, respectively. Survival of mice was monitored over time and is expressed as percent. Statistical comparison of survival curves using Log-ranked (Mantel-Cox) test showed a p-value of 0.0009.

FIGS. 4A-4E show graphs demonstrating the IgG antibody titer of the vaccinated and boosted BALB/c mice. One day prior to V, B, and Cl (open arrows in FIG. 1) venous blood from the tail was removed to obtain the basal, vaccine-induced, and boosting-induced anti-Salmonella IgG response, respectively. Twenty-five micrograms of NC1040 protein were added to each well, serum samples prepared from tail's venous blood one-day before vaccination, boosting and challenge were 2-fold serially diluted, and analyzed in duplicate. Data shown are the log2 of the reciprocal dilution. Statistical significance was determined by comparing the mean OD450 values against the before V values at each dilution. The last reciprocal dilution with mean OD450 values that were significantly different than the before V values were considered the endpoint dilution. The dotted line (1:100; FDR adjusted p value=0.031) and solid line (1:3,200; FDR adjusted p value=0.018) show the endpoint dilutions for the before V and before B doses, respectively. A multiple t-test with a 5% false discovery rate (FDR) post-hoc test with multiple comparisons was used to determine significance. Significance was determined by comparing the mean OD450 values against the pre-vaccination values (vaccination experiment #2).

FIG. 5 shows a graph demonstrating attenuation of the modified S. Typhimurium strain (vaccine strain) as compared to the parent virulent strain. Four 6-8 week-old female C57BL/6 mice were orally inoculated with a mixture containing 9.1×10⁶ of NC1190 (NC983-Rif^{R}) and 8×10⁶ of NC1040 (ATCC 14028s-Kan^{R}). At four days post infection (dpi), mice were euthanized and the bacterial burden in homogenized tissues was determined. The competitive index (CI; [24]) was calculated using the following equation: (NC1190_{OUT}/NC1040_{OUT})/(NC1190_{IN}/NC1040_{IN}). Each data point is the logo of the CI from a single mouse and tissue site.

FIG. 6 shows an image demonstrating detection of the anti-S. Typhimurium IgG response by immunoblotting. The equivalent of 2×10⁸ cells of whole-cell lysate from strain 14028s was loaded per lane and samples were separated by size on 15% acrylamide gels. Following transfer, membranes were blocked and probed with serum from individual BALB/c mice. Serum samples were obtained by tail bleeding at V (1 dpv), B (13 dpv), and Cl (34 dpv) to determine the host IgG response. Membranes were probed with secondary antibody (anti-mouse IgG conjugated to HRP) and detection of horseradish peroxidase activity was determined with 4-chloro-1-napthol and H₂O₂, as described in Materials and Methods. Arrows [symbol] , represent antigen-antibody complexes; Stared arrow [symbol] , represents early antigen-antibody complex.

FIGS. 7A-7B show images demonstrating a ponceau S stain for protein (FIG. 7A) and corresponding immunoblot (FIG. 7B) demonstrating expression of the heterologous OspC antigen that was cloned into the modified S. Typhimurium strain.

FIG. 8 shows the percent survival in mice vaccinated with S. Typhimurium strains that include single deletions of fnr or ynaF.

FIG. 9 shows a graph demonstrating the percent survival in mice vaccinated with an S. Typhimurium strain that includes a deletion of 24 of 26 candidate virulence genes.

FIG. 10 shows a cartoon summary of the various S. Typhimurium mutant strains examined.

FIG. 11 shows a graph demonstrating the results of an experiment to analyze the kinetics of systemic colonization and competitive fitness of strain NC983 and virulent S. Typhimurium. Six groups of 4 female C57BL/6 mice (a total of 24 mice) were inoculated with 5×10⁷ CFU of NC983. At 1, 2, 4, 8, 15, and 35 days post vaccination, 4 mice were euthanized and the bacterial burden of NC983 in the spleen (filled circles) and liver (open circles) was determined. Each point is an individual mouse and the mean±1 standard deviation is shown. The dash line indicates the limit of detection.

FIG. 12 shows a graph demonstrating the results of an experiment to analyze the kinetics of systemic colonization and competitive fitness of strain NC983 and virulent S. Typhimurium. Four groups of 4 C57BL/6 mice (a total of 16 mice) were inoculated with 5×10⁷ CFU of NC1189 (virulent Typhimurium). At 1, 2, 4, and 6 days, 4 mice were euthanized and the bacterial burden in the spleen (filled circles) and liver (open circles) tissue was determined as in FIG. 11. The dash line indicates the limit of detection.

FIG. 13 shows a graph demonstrating the bacterial burden of the challenge strain (NC1189) in the vaccinated mice at the termination of the study demonstrated in FIG. 2. Tissue samples were homogenized and plated on XLT4 agar plates containing 100 μg/mL of rifampicin and incubated at about 37° C. for 24 h to enumerate bacteria. Rif^{R} H₂S producing colonies were counted and are expressed as logo of the CFU/g of tissue sample.

FIG. 14 shows a graph demonstrating the production of anti-S. Typhimurium IgG in vaccinated mice. At the end of the challenge experiment outlined in FIG. 1, serum was collected from the surviving animals and assayed for anti-Salmonella IgG. The last reciprocal dilution with mean OD₄₅₀ values that were significantly different than the negative control serum was considered the endpoint dilution. The solid line shows the mean endpoint dilution (1:256,000; FDR adjusted p-value=0.044). A multiple t-test with a 5% false discovery rate (FDR) post-hoc test with multiple comparisons was used to determine significance. Significance was determined by comparing the mean OD₄₅₀ values against naïve litter mate controls (shown as a dotted line).

FIG. 15 shows a graph demonstrating the bacterial burden of the virulent challenge strain (NC1040) in vaccinated mice at termination of experiment #2. Tissue samples were homogenized and plated on XLT4 agar plates containing 65 μg/mL of kanamycin and incubated at 37° C. for 24 h to enumerate bacteria. Kan^{R} H₂S colonies were counted and are expressed as logo of the CFU/g of tissue sample.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

Definitions

As used herein, “about,” “approximately,” and the like, when used in connection with a numerical variable, generally refers to the value of the variable and to all values of the variable that are within the experimental error (e.g., within the 95% confidence interval for the mean) or within±10% of the indicated value, whichever is greater.

As used herein, “administering” can refer to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, by catheters, stents or via an implanted reservoir or other device that administers, either actively or passively (e.g. by diffusion) a composition the perivascular space and adventitia. The term “parenteral” can include subcutaneous, intravenous, intramuscular, intra-articular, intra-synovial, intrasternal, intrathecal, intrahepatic, intralesional, and intracranial injections or infusion techniques.

As used herein, “adjuvant” can refer to an additional compound, composition, or ingredient that can facilitate stimulation an immune response in addition to the main antigen of a composition, formulation, or vaccine. Generally, an adjuvant can increase the immune response of an antigen as compared to the antigen alone. This can improve and/or facilitate any protective immunity developed in the recipient subject in response to the antigen. “Adjuvant” as used herein can refer to a component that potentiates the immune responses to an antigen and/or modulates it towards the desired immune response(s).

The term “antibody” as used herein can refer to an immunoglobulin which specifically binds to and is thereby defined as complementary with a particular spatial and polar organization of another molecule. The antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, IgY, etc. Fragments thereof may include Fab, Fv and F(ab′)₂, Fab′, scFv, and the like. In addition, aggregates, polymers, and conjugates of immunoglobulins or their fragments can be used where appropriate so long as binding affinity for a particular molecule is maintained.

As used herein, “antigen” refers to a molecule with one or more epitopes that stimulate a host's immune system to make a secretory, humoral and/or cellular antigen-specific response, or to a DNA molecule that is capable of producing such an antigen in a vertebrate. The term is also used interchangeably with “immunogen.” For example, a specific antigen can be complete protein, portions of a protein, peptides, fusion proteins, glycosylated proteins and combinations thereof.

As used herein, “control” is an alternative subject or sample used in an experiment for comparison purpose and included to minimize or distinguish the effect of variables other than an independent variable.

As used herein, “deoxyribonucleic acid (DNA)” and “ribonucleic acid (RNA)” generally refer to any polyribonucleotide or polydeoxribonucleotide, which can be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers or coding mRNA (messenger RNA).

As used herein, “dose,” “unit dose,” or “dosage” can refer to physically discrete units suitable for use in a subject, each unit containing a predetermined quantity of a composition, formulation, and/or vaccine described herein.

As used herein, “effective amount” is an amount sufficient to effect beneficial or desired results. An effective amount can be administered in one or more administrations, applications, or dosages.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “engineered strain” can refer to a modified bacterial strain that can contain one or more structural (e.g. genetic, chemical, or otherwise) and/or functional modifications as compared to the wild-type strain.

As used herein, “gene” can refer to a hereditary unit corresponding to a sequence of DNA that occupies a specific location on a chromosome and that contains the genetic instruction for a characteristic(s) or trait(s) in an organism. “Gene” also refers to the specific sequence of DNA that is transcribed into an RNA transcript that can be translated into a polypeptide or be a catalytic RNA molecule including but not limited to tRNA, siRNA, piRNA, miRNA, long-non-coding RNA and shRNA.

As used herein, “gene deletion” can refer to a structural change (e.g. a point mutation, nucleotide addition and/or deletion) to the genome of an organism, including bacteria that results in a modulation in the function of a product produced from the region of the genome containing the structural change. The modulation can be a reduction, attenuation, elimination, or increase in the function and/or activity of the product produced form the region of the genome containing the structural change.

As used herein, “immune response” can refer to the reaction of the molecules, components, pathways, organs, fluids and/or cells of the body to the presence of a substance that is foreign or recognized by the body as foreign to the body.

As used herein, the term “immunization” can refer to the process of inducing a continuing protective level of antibody and/or cellular immune response which is directed against an S. enterica serovar, such as S. Typhimurium or antigen thereof, either before or after exposure of the host to a strain of S. enterica, such as S. Typhimurium, including but not limited to any one of the engineered S. Typhimurium strains described herein.

As used herein, “modulate or modulation of the immune response” can refer to change in the immune response that results from the introduction of a composition, vaccine, or other compound or formulation described herein in a recipient subject as compared to a suitable control.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{W}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “nucleic acid” and “polynucleotide” generally refer to a string of at least two base-sugar-phosphate combinations and refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, polynucleotide as used herein refers to triple-stranded regions comprising RNA or DNA or both RNA and DNA. The strands in such regions can be from the same molecule or from different molecules. The regions can include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotide” as that term is intended herein.

As used herein, “pharmaceutically acceptable carrier, diluent, binders, lubricants, glidant, preservative, flavoring agent, coloring agent, and excipient” refers to a carrier, diluent, binder, lubricant, glidant, preservative, flavoring agent, coloring agent, or excipient that is useful in preparing a pharmaceutical formulation that is generally safe, non-toxic, and is neither biologically or otherwise undesirable, and includes a carrier or excipient that is acceptable for veterinary use as well as human pharmaceutical use.

As used herein, “polypeptides” or “proteins” are amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, VV), Tyrosine (Tyr, Y), and Valine (Val, V).

As used herein, “preventative” refers to hindering or stopping a disease or condition before it occurs or while the disease or condition is still in the sub-clinical phase.

As used herein, the term “recombinant” generally refers to a non-naturally occurring nucleic acid, nucleic acid construct, or polypeptide. Such non-naturally occurring nucleic acids can include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a “fusion protein” (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter etc.). Recombinant also refers to the polypeptide encoded by the recombinant nucleic acid. Non-naturally occurring nucleic acids or polypeptides include nucleic acids and polypeptides modified by man, including but not limited to miRNA target sequences described herein.

As used interchangeably herein, “subject,” “individual,” or “patient,” refers to a vertebrate and/or a mammal. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

As used herein, “therapeutic” can refer to treating or curing a disease or condition.

As used herein, “vaccine” can refer to a compound, molecule, compositions, and formulations that are capable of inducing an immune response in a subject. The term “vaccine” can also be used to refer to a compound, molecule, compositions, and formulations that are capable of providing protective immunity against an organism. The vaccine may provide protection against a same (i.e. homologous) or different (i.e. heterologous) strain of an organism. The vaccine can be capable of providing protection against homologous and heterologous species, variants or strains.

As used herein, “wild-type” can refer the typical form of an organism, variety, strain, gene, protein, or characteristic as it occurs in nature, as distinguished from mutant forms that may result from selective breeding or transformation with a transgene.

Discussion

Salmonella is a bacterial pathogen that can cause a spectrum of human and animal diseases. Most salmonella infections are caused by food infected (contaminated) with S. enterica, which can infect cattle, poultry, and other domestic animals. Raw chicken and poultry eggs can also harbor S. enterica. Over 2,600 serovars have been identified for S. enterica, many of which are highly pathogenic to humans. Among the serogroups that cause the most human-related illnesses are S. Enteritidis, Typhi, and Typhimurium.

Current methods of reducing salmonella infection in farm animals are to prevent infection of the animals through good sanitation and hygiene practices. Efforts are made by farms and veterinarians to identify and isolate infected animals to prevent disease throughout a herd or flock.

Another methodology for reducing and preventing salmonella infection is to vaccinate animals, particularly those that may serve as transmission vectors for the bacteria. There are several vaccines currently available for S. Typhimurium, including Salmune, Poulvac ST, and Nobilis Salenvac T. The currently available vaccines for S. Typhimurium are based on a single gene mutation, which carries the risk of virulence returning from a compensatory mutation. As such, there exists a need for an improved vaccine that can at least provide protection against S. Typhimurium.

With that said, described herein are strains of S. Typhimurium that can have a deletion of more than one gene and that can have an attenuated virulence as compared to the unmodified strain. Also provided herein are vaccines that can contain modified S. Typhimurium bacteria that can have a deletion of more than one gene. In some embodiments, the S. Typhimurium strain can include a deletion of about 26 kb of the genome of the virulent parent strain. The strains of attenuated S. Typhimurium provided herein can provide a safer vaccination against S. Typhimurium as opposed to current vaccinations at least because there is a reduced possibility of compensatory mutations or reversion in the modified strain of S. Typhimurium. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Engineered Strain(s) of S. Typhimurium and Compositions Thereof

Provided herein are engineered strains of S. Typhimurium. The engineered strain of S. Typhimurium can include one or more gene deletions as compared to the wild-type S. Typhimurium (also referred to herein as the reference or parent strain). The wild-type S. Typhimurium serovar can be Salmonella enterica serovar Typhimurium 14028s, which can have a genomic sequence according to GenBank Accession No. NC_016855.1 (SEQ ID NO: 3) (Jarvik et al., 2010. J. Bacteriol 192:560-567) (SEQ ID NO: 3). The gene deletions of the engineered strain of S. Typhimurium can be genes residing between about nucleotides 1,736,853 and 1,764,155 of the wild-type S. Typhimurium. Table 1 lists various genes in the wild-type S. Typhimurium and their corresponding base pairs in the reference wild-type sequence.

TABLE 2

| Gene | Start bp | End bp |
|---|---|---|
| STM14_1981 | 1736853 | 1738403 |
| STM14_1982 | 1738867 | 1739529 |
| STM14_1983 | 1739800 | 1740330 |
| STM14_1984 | 1740447 | 1741247 |
| STM14_1985 | 1741311 | 1745156 |
| STM14_1986 | 1745414 | 1746019 |
| STM14_1987 | 1746016 | 1746465 |
| STM14_1988 | 1746608 | 1746931 |
| STM14_1989 | 1746939 | 1747130 |
| STM14_1990 | 1747130 | 1749766 |
| STM14_1991 | 1750006 | 1750995 |
| STM14_1992 | 1751106 | 1751516 |
| STM14_1993 | 1751560 | 1751715 |
| STM14_1994 | 1751742 | 1752041 |
| STM14_1995 | 1752102 | 1755626 |
| STM14_1996 | 1755685 | 1755894 |
| STM14_1998 | 1756592 | 1756930 |
| STM14_1999 | 1757094 | 1757234 |
| STM14_2000 | 1757286 | 1758221 |
| STM14_2001 | 1758265 | 1759638 |
| STM14_2003 | 1761254 | 1762408 |
| STM14_2004 | 1762419 | 1762580 |
| STM14_2005 | 1762819 | 1763382 |
| STM14_2006 | 1763640 | 1764155 |

The engineered strain of S. Typhimurium can include any single gene deletion of a gene, where the gene deleted can be selected from the following group of genes: STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1998, STM14_1999, STM14_2000, STM14_2001, STM14_2003, STM14_2004, STM14_2005, and STM14_2006.

The engineered S. Typhimurium strain can include any combination of 2,3,4,5,6,7,8,9, . . . 27 gene deletions, where the gene deleted can be selected from the following group of genes: STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997, STM14_1998, STM14_1999, STM14_2000, STM14_2001, STM14_2002, STM14_2003, STM14_2004, STM14_2005, STM14_2006, and STM14_2007, except for the combination of STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997 STM14_1998, STM14_1999, STM14_2000, STM14_2001, STM14_2002, STM14_2003, STM14_2004, STM14_2005, and STM14_2006.

In some embodiments, the engineered S. Typhimurium strain can include a deletion of STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997, STM14_1998, STM14_1999, STM14_2000, STM14_2001, STM14_2002, STM14_2003, STM14_2004, STM14_2005, and STM14_2006.

The engineered strains can be attenuated as compared to the wild-type S. Typhimurium.

Methods of genome modification (including nucleotide deletion and addition) are known to those of ordinary skill in the art. The engineered strains can be made and cultured using techniques of molecular biology, recombinant DNA technology, microbiology, and the like generally known to one of ordinary skill in the art.

Also provided herein are engineered S. Typhimurium bacteria, and/or compositions thereof as described herein, wherein the engineered S. Typhimurium bacteria previously described herein can be further engineered to include and/or express the foreign epitope. As used herein, “foreign epitope” can refer to an epitope that is considered non-self when compared to the subject that the foreign epitope is being delivered to. Methods of genome modification generally known in the art can be used to design and further modify the engineered S. Typhimurium bacteria to include and/or express a desired foreign epitope.

Also provided herein are whole cell compositions that can contain an engineered strain of S. Typhimurium described elsewhere herein. The compositions can contain an amount of one or more engineered S. Typhimurium strains described herein. The engineered S. Typhimurium strain(s) can be included as live bacteria in the composition. This can be possible as the engineered S. Typhimurium strain(s) can be attenuated as the result of the gene deletions. In some embodiments, the engineered S. Typhimurium strain(s) can be killed prior to inclusion in the composition. In embodiments, the compositions contain whole cell isolates of the engineered S. Typhimurium strains provided herein. Methods of killing bacteria for use in a composition, such as but not limited to a vaccine, are generally known in the art. Some of these methods include heat and chemical (e.g. by formaldehyde) killing. The engineered S. Typhimurium strains can independently be included in the composition in an amount or concentration ranging from about 10² cells per mL to about 10¹⁰ cells or more per mL. It will be appreciated that different amounts can be used or be effective in compositions, such as vaccines, for immunizing different species, which will be appreciated by one of ordinary skill in the art. Where two strains are included in the composition the ratio of each strain to each other can range from 1:1 to 10:1. It will be appreciated that the ratio of each strain can vary in effectiveness depending on species being immunized, which will be appreciated by one of ordinary skill in the art.

Vaccines

The compositions containing an engineered strain of S. Typhimurium strain(s) described herein can be formulated as vaccines. The compositions can be included in a combination vaccine or other combination formulation. In some embodiments, the combination vaccine or other combination formulation can include one or more engineered strain of S. Typhimurium strain(s) described herein as described herein and one or more additional killed and/or modified strain or isolate of another species or genus of bacterium, antigenic component of another species or genus of bacterium, killed or attenuated virus, antigenic component of a virus, and/or antibodies raised against another pathogenic organism.

The compositions and/or vaccines can contain an effective amount or concentration of one or more engineered S. Typhimurium strains described herein. The amount can be effective to stimulate an immune response, stimulate antibody production, provide protective immunity, immunize, treat, and/or prevent S. enterica, particularly the S. enterica serovar Typhimurium in the subject and/or offspring thereof. The effective amount or concentration of the one or more engineered strain of S. Typhimurium can range from about 10² cells per mL to about 10¹⁰ cells or more per mL. The compositions, vaccines, and/or other formulations described herein can be effective to stimulate an immune response, stimulate antibody production, and provide protective immunity against a strain of S. enterica serovar Typhimurium in the subject and/or offspring thereof. The effective amount or concentration of the one, immunize a subject against S. enterica serovar Typhimurium, treat and/or prevent any S. enterica serovar Typhimurium infection in the subject and/or offspring thereof. In embodiments, the subject can be a chicken, other avian species, or other domestic farm animal species.

The vaccines can contain one or more additional ingredients. The vaccines can include one or more suitable adjuvants. Suitable adjuvants are generally known in the art and can include, but are not limited to aluminum salts (e.g, aluminum phosphate and aluminum hydroxide), organic adjuvants (e.g. squalene), and oil-based (e.g., MF59). In embodiments, the vaccines can contain a suitable pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxyl methylcellulose, and polyvinyl pyrrolidone, which do not deleteriously react with the active composition.

The vaccines can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition. In some embodiments, the vaccine can be produced under clean and/or sterile conditions. In some the vaccine can be produced under clean and/or sterile conditions but is not necessarily sterilized.

In addition to the engineered S. Typhimurium strain(s), the vaccines can also include an amount, including an effective amount, of one or more of auxiliary active agents, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

Methods of Treating and/or Preventing S. enterica serovar Typhimurium Infection

The compositions, and vaccines provided herein can be administered to a subject. The subject can be a chicken other avian species, other domestic farm animal species, or human subjects (including, but not limited to farm workers and the public). The chicken can be a chicken 2 weeks or older. The chicken can be a chicken less than two weeks of age. In some embodiments, the subject is a late stage embryo. In embodiments, the chicken can be a hen that is producing eggs. Administration of a composition and/or vaccine provided herein can induce or otherwise stimulate an immune response in the recipient subject and/or an offspring of the recipient subject. Administration of a composition, and/or vaccine provided herein can stimulate antibody production in the recipient subject. In other embodiments, administration of a composition and/or vaccine provided herein can provide protective immunity against S. enterica serovar Typhimurium in a recipient subject and/or an offspring of the recipient subject. Administration of a compound, composition, formulation, and/or vaccine provided herein can to a subject can treat/and or prevent S. enterica serovar Typhimurium infection in the recipient subject and/or offspring thereof.

Accordingly, provided herein are methods of inducing or otherwise stimulating an immune response in a subject and/or an offspring of the subject that include the step of administering a compound, composition, formulation and/or vaccine to a subject one or more times. Also provided herein are methods of stimulating antibody production in a subject and/or offspring of the subject that includes the step of administering a composition and/or vaccine to a subject one or more times. Also provided herein are methods of stimulating protective immunity a subject and/or offspring thereof by administering a composition and/or vaccine to a subject one or more times. Also provided herein are methods of treating and/or preventing S. enterica serovar Typhimurium infection by administering a composition and/or vaccine to a subject one or more times. In embodiments, the amount of the composition and/or vaccine can be an amount effective to stimulate an immune response, stimulate antibody production, provide protective immunity, immunize, treat, and/or prevent S. enterica serovar Typhimurium in the subject and/or offspring thereof.

The compositions and/or vaccines provided herein can be administered to the subject by any suitable route(s). In addition to the other suitable routes described elsewhere herein, the compositions and/or vaccines can be administered by water supply, aerosol mist, and/or vapor that can be applied through a misting system configured for vaccine delivery to multiple chickens, and/or by in ovo injection. Other suitable routs of administration include any other route generally used for delivery of vaccines and other compositions to chickens, avians, and other domestic farm animals. Such methods and routes of administration will be appreciated by those of ordinary skill in the art. In some embodiments, 0.01 cc to 10 cc or more of the composition and/or vaccine can be administered to a subject. It will be appreciated by those of skill in the art will depend on, inter alia, the size of the subject, the species of the subject, and the concentration of cells in the formulation being administered. In some embodiments, an amount effective to induce an immune response against S. enterica serovar Typhimurium in the recipient subject and/or offspring thereof.

The compositions and/or vaccines provided herein can be administered to subject one or more times. Where administration occurs more than once the time period between each does can each independently range from days (e.g. 1-7 days), weeks (e.g. 1-52 weeks, or years (e.g. 1-5 years) apart. Administration can occur during any life stage of the subject. Where the subject is a chicken or other avian, administration can, in some embodiments, occur in ovo, (e.g. 3-5 days before hatch), during the early post-hatch period (e.g. during the first two weeks post hatch), and during egg production. Administration can be simultaneously or in series with other vaccines.

Also provided herein are methods of delivering a foreign epitope to a subject in need thereof. As used herein, “foreign epitope” can refer to an epitope that is considered non-self when compared to the subject that the foreign epitope is being delivered to. The method can include administering engineered S. Typhimurium bacteria and/or composition thereof described herein to a subject in need thereof, wherein the engineered S. Typhimurium bacteria can be further engineered to include and/or express the foreign epitope as described elsewhere herein.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

### Complete Genome Sequence of a Salmonella enterica serovar Typhimurium Live, Attenuated Strain

Strain NC983 is highly attenuated in mice (1, unpublished data). NC983 was generated through fusaric acid-mediated removal of the tetracycline marker (2) from an fnr::Tn10 mutation in S. Typhimurium ATCC 14028s. DNA was extracted from lysed bead beaten (Biospec Products; Bartlesville, Okla.) NC983 cells using a FastDNA™ SPIN Kit for Soil (MP Biomedicals; Santa Ana, Calif.). Eluted DNA was concentrated and processed with the PacBio whole-genome sequencing workflow using the Pacific Biosciences RS II sequencing platform (Pacific Biosciences, Menlo Park, Calif.). The 20-kb SMRTbell™ Templates kit was used for template preparation. The library was prepared using a 10-kb template library preparation workflow from size-selected templates (BluePippin™ V3 Cassette Definition for 10,000 bp). Three SMRT cells were used on a PacBio RS II sequencer with the C4 sequencing chemistry and P6 polymerase. The Ion Torrent reads were obtained from fragmented DNA and libraries were prepared and purified using AMPure beads. Specific adapters Ion P1 and Ion Xpress™ Barcode X were ligated to fragmented DNA using the Ion Plus Fragment Library and Barcode Adapters Kits (Life Technology, Thermo Fisher Division, Waltham, Mass.). Ligated DNA was nick repaired, purified, size-selected, and amplified. DNA templates were sequenced on an Ion Torrent PGM using Ion PGM300 sequencing reagents (Life Technology). Base pair calling and sequence trimming were performed on the Ion Torrent browser.

The PacBio continuous long reads were error corrected using the Hierarchical Genome Assembly Process (HGAP) workflow (PacBioDevNet; Pacific Biosciences; SMRT Analysis version 2.2) and a de novo assembly of the corrected reads was conducted using MIRA version 4.0.2 (3). The resulting assembly (48×) consisted of six contigs, two of which were large non-repetitive contigs. Ion Torrent reads (19×) were mapped to the alignment of the two large contigs using MIRA to increase the average consensus quality. The resulting consensus contigs were circularized using the Minimus 2 assembler (4) and polished using Quiver (5).

The longer of the two contigs mapped to the chromosomal reference sequence of 14028s (NC_016856.1; 4,870,265 bp) and the shorter of the two contigs mapped to the plasmid reference sequence of 14028s (NC_016855.1; 93,832 bp) (6). Five G/C homopolymer runs in protein coding regions within the chromosomal contig were corrected by adding either a single G or C to match that of the 14028s reference. After circularization and polishing, the chromosomal DNA was 4,846,304 bp in length (average PacBio base coverage: 298×) and the virulence plasmid, pLST, was 93,829 bp in length (average PacBio base coverage: 461×).

The NCBI Prokaryotic Genome Annotation Pipeline (available online) was used for annotation and it identified 4,612 protein coding-genes with 85 tRNAs, 8 5S, 7 16S, and 7 23S rRNA genes. Strain NC983 contains a large deletion that removed base pairs 1,737,878 to 1,764,448 from the genome of 14028s (6). This stretch of sequence in 14028s has been replaced in NC983 with a 1,332 bp remnant of the Tn10 transposable element. The complete genome and virulence plasmid sequences of NC983 were deposited in GenBank with Accession numbers CP015157 (SEQ ID NO: 1) and CP015158 (SEQ ID NO: 2), respectively.

### References for Example 1

1. Fink R C, Evans M R, Porwollik S, Vazquez-Torres A, Jones-Carson J, Troxell B, Libby S J, McClelland M, Hassan H M. 2007. FNR is a global regulator of virulence and anaerobic metabolism in Salmonella enterica serovar Typhimurium (ATCC 14028s). J Bacteriol 189:2262-2273.
2. Bochner B R, Huang H C, Schieven G L, Ames B N. 1980. Positive selection for loss of tetracycline resistance. J Bacteriol 143:926-933.
3. Chevreux B, Wetter T, Suhai S. 1999. Genome Sequence Assembly Using Trace Signals and Additional Sequence Information. Computer Science and Biology: Proceedings of the German Conference on Bioinformatics (GCB) 99: 45-56.
4. Sommer D D, Delcher A L, Salzberg S L, Pop M. 2007. Minimus: a fast, lightweight genome assembler. BMC Bioinformatics 8, 64.
5. Chin C S, Alexander D H, Marks P, Klammer A A, Drake J, Heiner C, Clum A, Copeland A, Huddleston J, Eichler E E, Turner S W, Korlach J. 2013. Nonhybrid, finished microbial genome assemblies from long-read SMRT sequencing data. Nat Meth 10:563-569.
6. Jarvik T, Smillie C, Groisman E A, Ochman H. 2010. Short-term signatures of evolutionary change in the Salmonella enterica serovar Typhimurium 14028 genome. J Bacteriol 192:560-567.

### Example 2

### Vaccination With Attenuated S. Typhimurium Strains

Background. Foodborne pathogens are a group of infectious agents that threaten public health. An example of this is the non-Typhoidal Salmonella enterica (NTS) serovars. NTS have been linked to the majority of bacterial foodborne outbreaks within the U.S.A. [1]. Among NTS, the serovar S. Typhimurium has been one of the most frequently identified by the Center for Disease Control (CDC). According to the CDC, this serovar was implicated in two outbreaks/year during 2014, 2013, and 2012, three outbreaks in 2011, and one in 2009 and 2006.

Early work determined that a live attenuated strain generates superior protection against wild-type challenge compared to heat inactivation of a virulent strain [2]. Subsequently, several strategies were employed to attenuate Salmonella including mutations affecting: metabolic functions (i.e., auxotrophic mutants), virulence genes, LPS biosynthesis, or other regulatory elements [Reviewed in 3-5]. Thus, mutants of S. enterica strains bearing defects in aromatic amino acid biosynthesis (e.g., aroA and aroD) and nucleic acid biosynthesis (e.g., purA and purE), as well as, mutations in the UDP-glucose 4-epimerase (galE) functioned as live-attenuated vaccines for multiple Salmonella serovars [6-12]. However, auxotrophic mutants have the potential for a reversion of virulence that is partially influenced by the host's diet [6,13]. Furthermore, recent findings showed ΔaroA mutants to be metabolically and physiologically pleotropic, and introduction of ΔaroA in an attenuated strain of Salmonella increased its virulence in mouse models [14]. S. Typhimurium strains deleted in the lipoprotein genes (IppAB) alone or in combination with an acetyltransferase gene (msbB), that is required for the modification of lipid-A in LPS, provided protective immunity in mice [15]. A recent systematic review and meta-analysis of 126 published studies describing the use of Salmonella vaccines in swine (majority were S. Typhimurium and S. Choleraesuis strains) concluded that the use of vaccines was beneficial but variable [16]. Clearly, the search for better Salmonella vaccines for use in farm animals and protect the consumers is far from over.

Earlier work showed that strain NC983 is attenuated in mice [17]; however, its utility as a live attenuated vaccine strain was not contemplated or tested. Strain NC983 is derived from the highly virulent strain 14028s (American Type Culture Collection strain, ATCC 14028s; a smooth-colony variant derived from CDC60-6516) that was isolated in 1960 from samples of hearts and livers of 4-week-old chickens [18, 19]. Strain NC983 contains a large deletion that removed base pairs 1,737,878 to 1,764,448 from the genome of 14028s [20, Example 1]. Because of this large deletion, it is less likely that this strain will undergo a reversion to virulence within the host. This genetic region is conserved within the S. enterica genomes sequenced to date.

In this Example, data is presented that can demonstrate that strain NC983 can be a vaccine. Strain NC983 was observed to be immunogenic as evidenced from induction of an anti-Salmonella immunoglobulin (IgG) response. In addition, competition experiments demonstrated that strain NC983 exhibited a profound decrease in fitness within the spleen (about 4 orders of magnitude) compared to the parental virulent strain. Strain NC983 protected Salmonella sensitive (Ity^{s}; C57BL/6 and BALB/c) mouse backgrounds from wild-type challenge.

Materials and Methods.

Bacterial Strains. Table 2 lists the bacterial strains used in this Example. The parental strain used in this study is from ATCC. Construction of spontaneous rifampicin resistant Typhimurium strains were generated as described previously [21]. Strain NC1040 is a kanamycin resistant derivative of 14028s that is fully virulent in mice and was constructed as described previously [21].

TABLE 2

| Strain | Genotype^{a} | Source |
|---|---|---|
| Salmonella enterica serovar | Wild-Type | ATCC^{b} |
| Typhimurium 14028s | | |
| NC983 | Fusaric Acid Resistant | [17] |
| NC1040 | ATCC 14028s fnr′:ha (Kan^{R}) | [21] |
| NC1189 | ATCC 14028s (Rif^{R}) | This |
| | | Example |
| NC1190 | NC983 (Rif^{R}) | This |
| | | Example |

^{a}Rif^{R} (rifampicin resistant) and Kan^{R} (kanamycin resistant)
^{b}ATCC (American Type Culture Collection)

Bacterial Growth and Preparation of Cell Suspensions. NC983 or the challenge strains were grown overnight at 37° C. in about 100 ml of Luria-Bertani (LB; 10 g tryptone, 5 g yeast extract, and 10 g NaCI per L) under static culture conditions. Bacteria were centrifuged, washed in phosphate buffered saline (PBS), and resuspended in a small volume (about 3 mL) of PBS. The optical density at 600 nm (OD₆₀₀) of the concentrated cell suspension was determined using a BioRad Smartspec 3000 with a 1 cm light path, and adjusted, according to a standard predetermined relationship between OD₆₀₀ and viable cell counts (i.e., 1 OD₆₀₀ about 1×10⁹ CFU/mL), to an appropriate cell density as indicated in the results. The cell suspension was diluted and plated to confirm the actual viable CFU/ml.

Animals. Six to eight week old C57BL/6 and BALB/c (Ity^{s}, both strains are S. Typhimurium sensitive) female mice from Jackson Laboratories (Bar Harbor, Me.) and Harlan Lab (now Envigo, Indianapolis, Ind.), respectively, were used. Mice were housed in disposable cages (3-4 mice per cage) and had access to sterile water and food (PicoLab Mouse Diet 2) ad libitum.

Determination of dose required to kill 50% of mice (Wa). The lethal dose required to kill 50% of animals (LD₅₀) for S. Typhimurium ATCC 14028s was determined under our conditions. Four groups of mice (4-mice per group) each received an oral dose of about 3.5×10¹, about 3.5×10², about 3.5×10³, or about 3.5×10⁴ CFU/mouse. Mice were monitored for about 14 days and the LD₅₀ was calculated from 10 day survival data according to [22] and [23]. The LD₅₀ was about 10³ CFU per C57BL/6 mouse.

Fitness of NC983 in vivo. To determine the ability of NC983 to colonize different tissues, groups of 3-4 C57BL/6 female mice (aged 6-8 weeks) were inoculated with about 5×10⁷ CFU/mouse of either the parental strain (14028s) or the vaccine strain (NC983). Mice were euthanized at indicated time points and viable S. Typhimurium within the spleen and liver were determined as described above. In another experiment, the competitive index (CI; [24) for NC983 Rif^{R} (i.e., NC1190) and the virulent 14028s Kan^{R} (i.e., NC1040) was determined. Four mice, C57BL/6 as above, were given an oral dose of about 9.1×10⁶ and about 8×10⁶ of NC1190 and NC1040, respectively. At four days post infection (dpi), mice were euthanized and the bacterial burden in homogenized tissues was determined by plating each sample on XLT4 agar plates containing rifampicin (to enumerate NC983), and XLT4 agar plates containing about 65 μg/mL kanamycin (to enumerate 14028s).

Vaccination and Challenge Protocols.

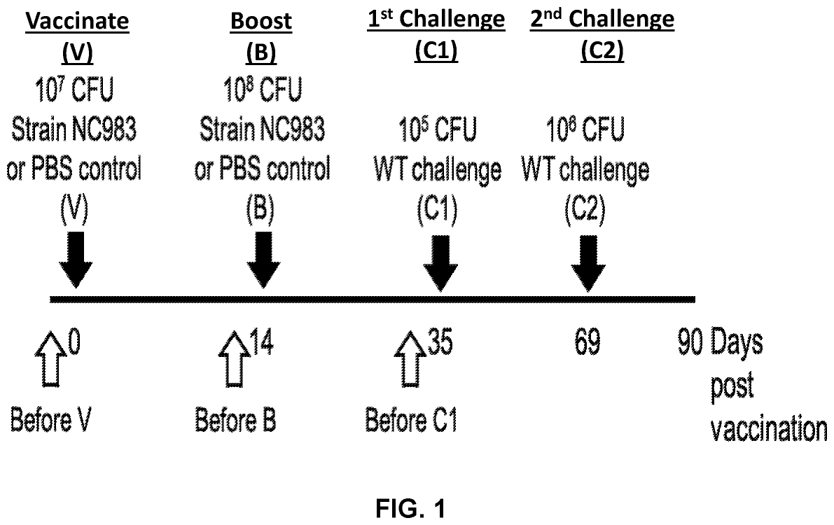

The mice were subjected to the vaccination protocol shown in FIG. 1. The vaccination and boosting doses were determined in preliminary studies. Each vaccinated or challenged mouse received a 100 μL of the appropriate cell suspension (see above) by oral gavage. Control mice received an equal volume of the PBS solution.

In vaccination experiment #1, C57BL/6 mice were given a vaccination dose (about 10⁷ CFU/mouse) and at 14 days post vaccination (dpv) they received a boosting dose (about 10⁸ CFU/mouse). In earlier experiments, the vaccination doses ranged from about 1×10⁷ to about 5×10⁷. Boosting doses were administered at varying times post initial vaccination (between 8 and 15 days post initial vaccination). The boosting dose was varied between about 5×10⁶ to about 1×10⁸. Based on animal body condition score and IgG titer, that vaccine and boosting doses of about 10⁷ and about 10⁸, respectively, were optimal for the immune response although positive results were observed at other doses (data not shown). At 21 days post boosting, (equals 35 dpv) all mice were challenged with a dose of 100× the LD₅₀ (about 10⁵ CFU/mouse) of the virulent S. Typhimurium strain (NC1189 (Rif^{R}) and disease symptoms were monitored using the body condition scoring (BCS) as described [25]. A BCS score of 2 indicates that the animal is under-conditioned and is considered moribund. A BCS score of 2 is observed in mice that exhibit segmentation of vertebral column with detectable pelvic bones. A BCS score of 4-5 indicates a healthy mouse that does not exhibit lack of grooming, eating/drinking, nesting, and other functions of active mice. Mice that survived until 69 dpv were re-challenged with a higher dose, 1,000×LD50 (i.e., 10⁶ CFU/mouse). At 90 dpv (i.e., 55 and 21 days post first and second challenges, respectively), all mice were euthanized, blood for analysis of the anti-Salmonella IgG response was obtained by cardiac puncture. Cardiac puncture blood was collected at the end of vaccine experiment #1 in Sarstedt micro tube 1.1 ml Z-gel (Fisher Scientific, catalog #50-809-211), samples were treated, and serum collected according to the supplier instructions. The bacterial burden of the challenge strain (NC1189) was determined within the colon, spleen, and liver following homogenization of tissues and plating on buffered XLT4-MOPS agar plates containing 100 μg/mL rifampicin as described previously [21].

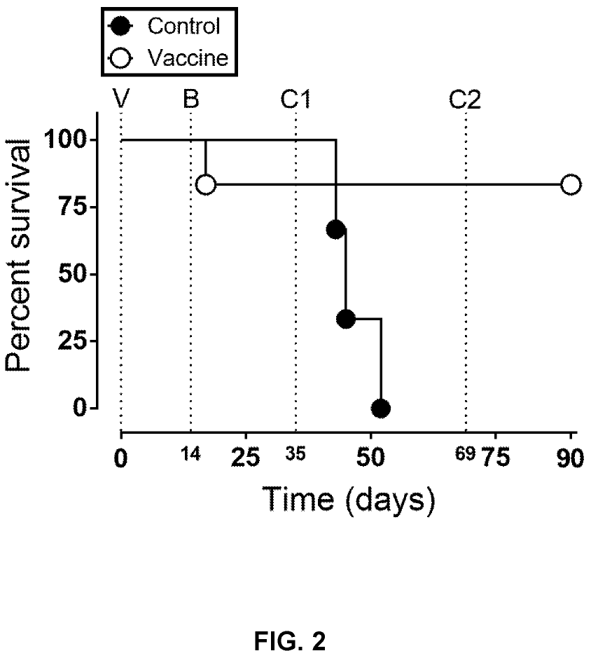

In vaccination experiment #2 (BALB/c) mice were given the vaccine, boost, and challenge doses (C1 and C2) as described above and in FIGS. 2 and 13-14. To measure the antibody response in BALB/c mice to the vaccine strain NC983 in a longitudinal manner, venous blood was obtained through tail bleeding at one day prior to vaccination (before V), boosting (before B), and the 1^{st} challenge (C1; before C1; FIG. 1).

Measurement of the anti-Salmonella IgG response by ELISA. Venous or cardiac puncture blood was allowed to clot at room temperature (about 20 minutes) before centrifuging at 20,000×g for 15 minutes at 4° C. and the supernatant (i.e., serum) was used to measure the anti-Salmonella IgG response.

To determine the end-point titers for detection of anti-Salmonella antigen, strain 14028s Kan^{R} (i.e., NC1040) was grown without shaking (still) overnight in LB containing 20mM glucose (glucose was added to improved cell yield). Cells were centrifuged, washed with PBS, concentrated in PBS followed by 60-cycles of sonication (each cycle was about 15 seconds on and about 30 seconds off and 60 cycles combined for a total sonication time of about 15 minutes) using a 20 KHz Heat Systems-Ultrasonics, Inc sonicator, model W-370—set at about 50% of its max output. Samples were kept on ice during and between rounds of sonication. The cell debris was removed by centrifugation at about 20,000×g for 15 minutes and the supernatant (cell-free extract, CF-Ext) was used as the Salmonella antigen. The protein concentration in the CF-Ext was determined using the Biorad Protein Assay Dye Reagent Concentrate according to manufacturer's specifications (Biorad; Hercules, Calif.).

Proteins from the cell-free extracts were diluted in ELISA coating buffer (about 50 mM carbonate-bicarbonate, pH 9.6; Sigma-Aldrich, St. Louis, Mo.) to about 250 μg/mL. One hundred μL of the solution was added to each well (about 25 μg) of a Corning 96-well EIA/RIA clear flat bottom polystyrene microplate (product #3361) and the plate was incubated overnight at about 4° C. The following day the solution in each well was removed and wells were washed three times with about 200 μL wash solution (about 50 mM Tris base, about 0.14 M NaCl, about 0.05% Tween 20, about pH 8.0). After washing, the wells were blocked for 15 minutes with the addition of about 200 μL of Super Block (ScyTek Laboratories, Inc; Logan, Utah). Serum samples from mice were 2-fold serially diluted in antibody buffer (about 50 mM Tris, about 0.14 M NaCl, 1% BSA) and about 100 μL of each dilution were added to wells in duplicate. Plates were incubated at room temperature for about 2 hours and washed as described above. Secondary antibody (Rabbit anti-mouse IgG (H+L) conjugated to HRP; Southern Biotech, Birmingham, Ala.) was diluted in antibody buffer to 1: about 10,000 and about 100 μL was added to each well. Plates were incubated at room temperature for about 2 hours and washed as described above. About 100 μL of HRP substrate, 1-Step™ Ultra TMB-ELISA Substrate Solution (ThermoFisher Scientific; Waltham, Mass.), was added to each well and incubated at room temperature for about 15 minutes. The reaction was terminated by the addition of about 100 μL of 2 M H₂SO₄ and the absorbance at 450 nm was recorded with a multi-mode plate reader (BioTek Synergy HTX; BioTek Instruments, Inc, Winooski, Vt.). Mean absorbance values were plotted against the Log₂ of the reciprocal dilution. A multiple t-test with a 5% false discovery rate (FDR) post-hoc test with multiple comparisons was used to determine significance. Significance was determined by comparing the mean OD₄₅₀ values of naïve litter mate controls (vaccination experiment #1) or against the pre-vaccination values (vaccination experiment #2). Figures and statistical analysis were accomplished using GraphPad Prism v7.03.

Measurement of the anti-Salmonella IgG response by immunoblot. Strain 14028s was grown as described above, centrifuged, washed, and the cell pellets were suspended in Laemmli sample buffer. Samples were denatured by boiling. Approximately 2×10⁸ cells were loaded per lane and samples were separated by size on 15% acrylamide gels (SDS-PAGE) and transferred to about 0.2 μM nitrocellulose membranes (Bio-Rad, Hercules, Calif.). Immunoblotting was performed as described previously [26, 27]. Briefly, membranes were stained with Ponceau S (about 0.1% Ponceau S (w/v), 1% acetic acid) to ensure equivalent loading of samples. For immunoblotting, membranes were blocked in a blocking buffer (PBS containing about 0.05% Tween-20 and about 1% powered non-fat milk, about pH 7.4) and probed with serum from BALB/c mice (primary antibody at about 1:1,000 for about 3 h). Membranes were washed 3 times with the blocking buffer and probed with secondary antibody (peroxidase-conjugated goat anti-IgG mouse antibody; Jackson ImmunoResearch Laboratories; West Grove, Pa.) at about 1:5,000 for about 3 h. Membranes were washed 3 times with Tris-NaCl (about 50 mM Tris, about 200 mM NaCl, about pH 7.6) and detection of horseradish peroxidase activity was determined in Tris-NaCl using 4-chloro-1-napthol (4CN; dissolved in methanol) and H₂O₂ (Thermo Fisher Scientific; Waltham, Mass.).

Statistical Analysis.

For survival plots, Log-ranked (Mantel-Cox) test was applied using Graph Pad Prism v. 7.03. For statistical analysis of anti-Salmonella IgG, a multiple t-test with a 5% false discovery rate (FDR) post-hoc test with multiple comparisons (Graph Pad Prism v. 7.03) was used to determine significance. In all cases p-values<0.05 were considered significant.

Results.

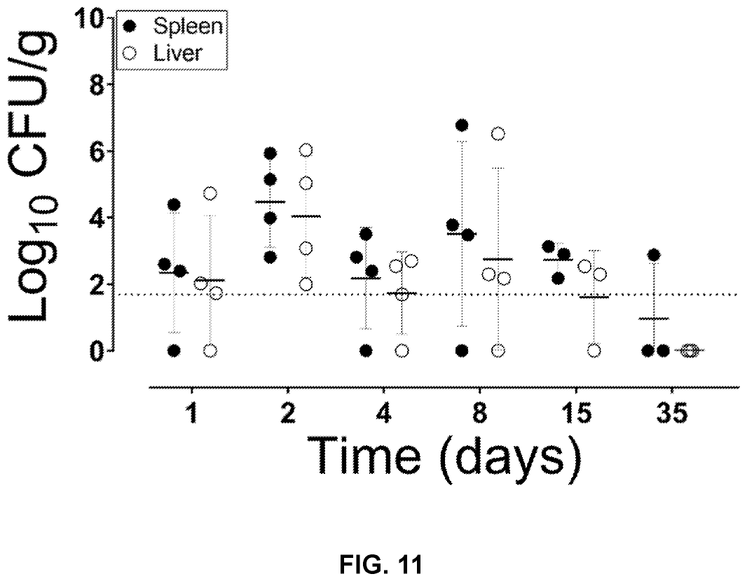

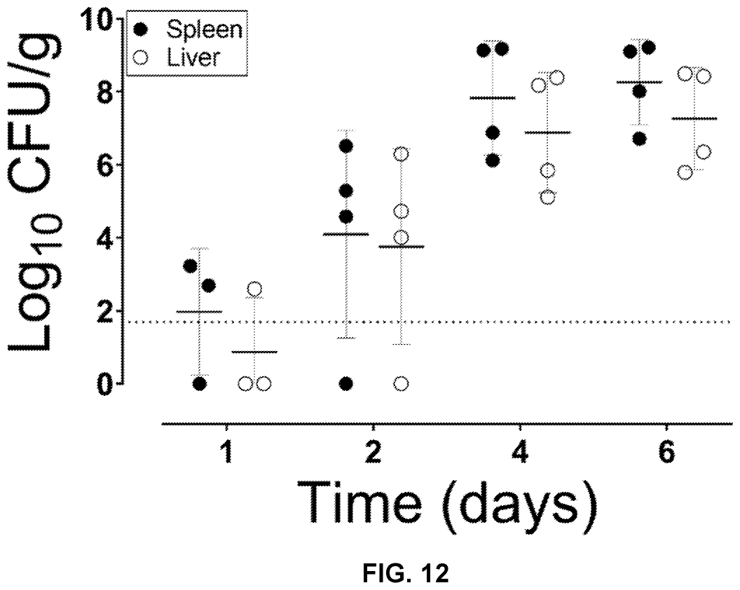

Strain NC983 exhibits a fitness defect in the colonization of the spleen. The kinetics of liver and spleen colonization for strain NC983 and the challenge virulent strain 14028s -Rif^{R} (NC1189) was determined. At 1 dpv, 3 out of the 4 mice had detectable levels of NC983 in the spleen and liver tissues (FIG. 11). At 2 dpv, all mice had quantifiable levels of NC983, but at 4, 8, and 15 dpv there was at least one mouse at each time point with undetectable levels of NC983 (FIG. 11). At 35 dpv, one mouse had detectable NC983 in the splenic tissues (FIG. 11. On the other hand, the kinetics of liver and spleen colonization by the virulent challenge strain, 14028s-Rif^{R} (NC1189), showed a different pattern (FIG. 12). At days 1, 2, 4, and 6 post infection, mice were euthanized and the bacterial burden was determined. By 4 dpi, all mice had concentrations of the challenge strain that were >10⁴ CFU/g tissue; and at 6 dpi, concentrations of 14028s-Rif^{R} (NC1189) reached about 10⁷ CFU/g in all mice (FIG. 12). No further time-point data were collected because the mice had a body condition score (BCS) about 2 and were euthanized.

Clearly, at days 1 and 2 post inoculation the kinetics of colonization of the liver and spleen by the vaccine strain was like that of the wild-type parent strain. However, at beyond 4 dpv, the vaccine strain showed much weaker colonization of the liver and spleen than the wild-type. This finding was confirmed by the data from the Competitive Index study (FIG. 5) In this type of assay, the fitness of the vaccine strain in the different murine tissues is simultaneously compared to that of the wild-type strain in the same animal [24] and FIG. 5. The Logo of the competitive index (CI) for the vaccine strain (NC1190) versus the challenge strain NC1040 was variable between the different animals in the colon and liver tissue sites, with an average fitness defect of about 2 orders of magnitude (FIG. 5). However, within the spleen for all mice there was a clear fitness defect of about 4 orders of magnitude (i.e., 10,000-fold reduction) between the vaccine strain compared to the virulent strain (NC1040) (FIG. 5). The kinetics and CI data (FIGS. 5 and 11-12) indicated that strain NC983 has a general fitness defect in the mice, but showed a clear and profound defect within the spleen.

Strain NC983 is a live attenuated Salmonella strain that protects against virulent S. Typhimurium and is immunogenic in mice. Previous work demonstrated that strain NC983 was unable to cause lethal infection in C57BL/6 mice when inoculated through either peroral or intraperitoneal routes [17]. This evidence suggested that NC983 maybe attenuated in mice and further studies were needed to test its ability to confer protective immunity in mice. Therefore, a vaccination protocol was developed to test the ability of NC983 to protect against challenge with virulent S. Typhimurium (FIG. 1). This protocol utilized oral inoculation (i.e., vaccination and boost) of mice with either strain NC983 (vaccine group) or a PBS control. At 35 days post-vaccination (dpv), all mice were challenged with the virulent strain of S.Typhimurium ATCC 14028s, as outlined in Materials and Methods and FIG. 1. The percent survival of mice was recorded for the duration of the study, bacterial burden of the challenge strain in vaccinated mice and anti-Salmonella IgG in vaccinated mice were determined at the end of the study (FIGS. 2 and 13-14).

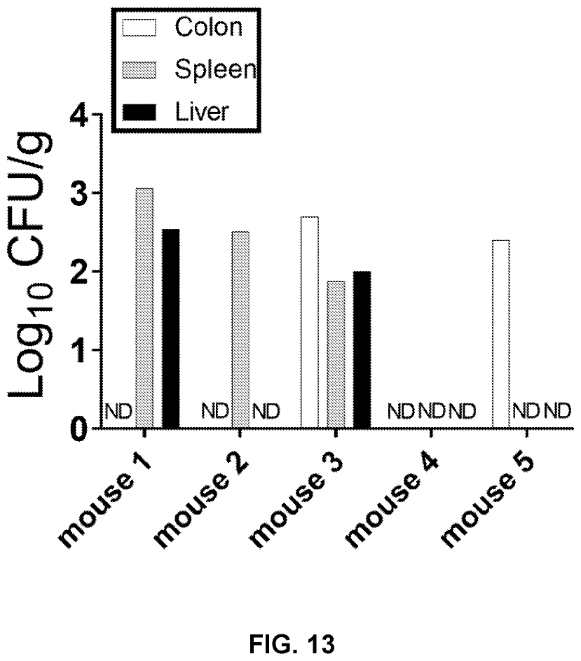

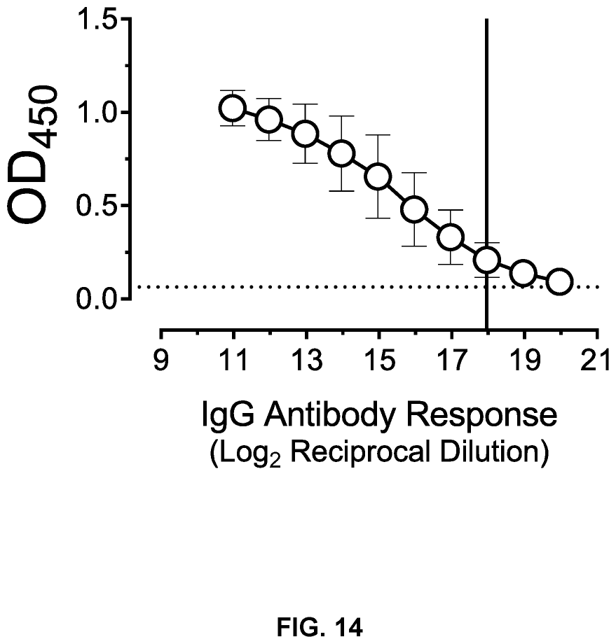

The data showed that by day 17 post challenge, all control mice (n=3) had died or required euthanasia (FIG. 2). Although one mouse of the vaccinated group was found dead after the boosting, the remaining mice (n=5) exhibited 100% survival post the two challenges (FIG. 2). At 90 dpv, all mice were euthanized and samples were processed to determine the bacterial burden of the WT virulent S. Typhimurium strain in the vaccinated mice (FIG. 13). The level of the vaccine strain (NC983) was undetectable in all tissues and the colon of these mice (data not shown). However, the challenge strain was found in quantifiable levels in colon samples from two mice. In addition, three splenic samples and two liver samples exhibited levels of the challenge strain between 10² and 10³ CFU/g (FIG. 13). When the anti-S. Typhimurium IgG levels were measured from these mice at 90-dpv, the mean endpoint titer was 1:256,000 (FDR adjusted p value=0.044; FIG. 14).

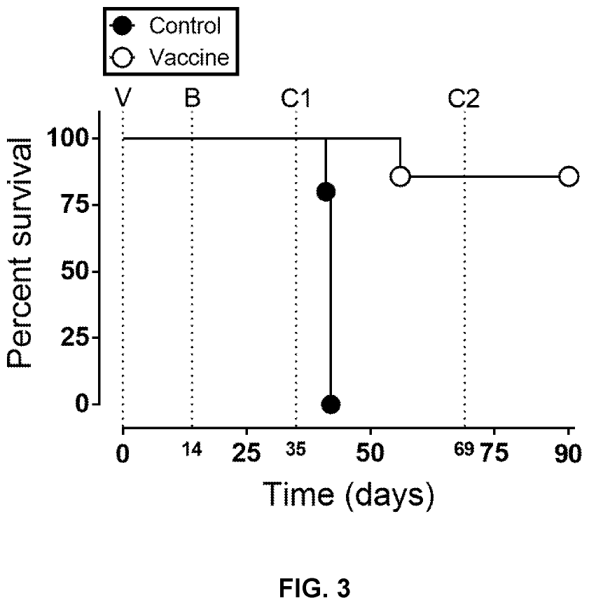

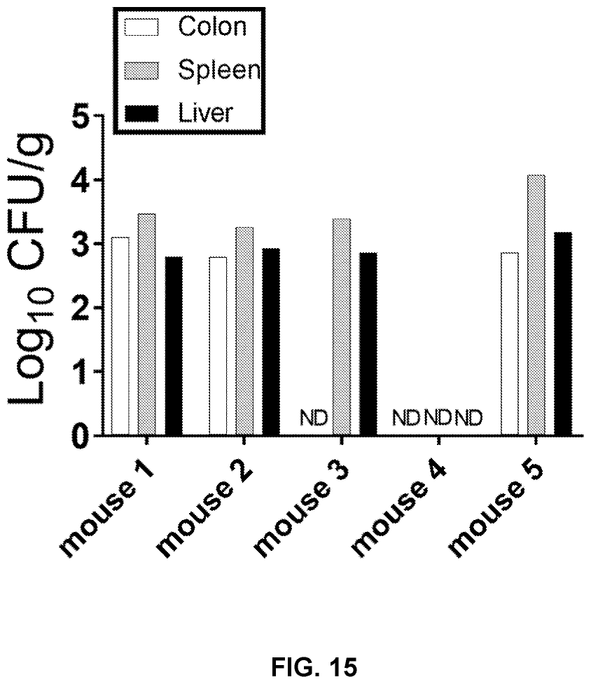

The vaccination protocol was repeated (vaccine experiment #2) with another Salmonella sensitive strain of mice (BALB/c) to ensure results from C57BL/6 were not strain specific. In this experiment, a preliminary LD₅₀ for the BALB/c showed that it was slightly lower than that of the C57BL/6 mice. However, to be on the safe-side, we used the same LD₅₀ as that for the C57BL/6. Also, the responses of individual mice were measured over time in a longitudinal approach (FIGS. 3 and 15). At 35 dpv, the BALB/c mice received on oral dose of the virulent strain, NC1040 (6.4×10⁴ CFU/mouse). By 7 days post challenge (C1, or 42 dpv), all control mice had died or required euthanasia (FIG. 3). At 21 days post challenge (56 dpv), one vaccine group mouse had to be euthanized; however, the remaining five mice survived another challenge dose (C2; 1.2×10⁶ CFU/mouse) (FIG. 3). At the end of the experiment (90 dpv), the bacterial burden of the challenge strain NC1040 was determined. Three mice (mouse 1, 2, and 5) had detectable levels of the challenge strain in all three examined tissues (colon, spleen, and liver). However, we did not detect the challenge strain in the colons of mouse 3 or 4. In addition, mouse 3 contained the challenge strain in the spleen and liver tissues, but mouse 4 had no challenge strain in any examined sites (FIG. 15).

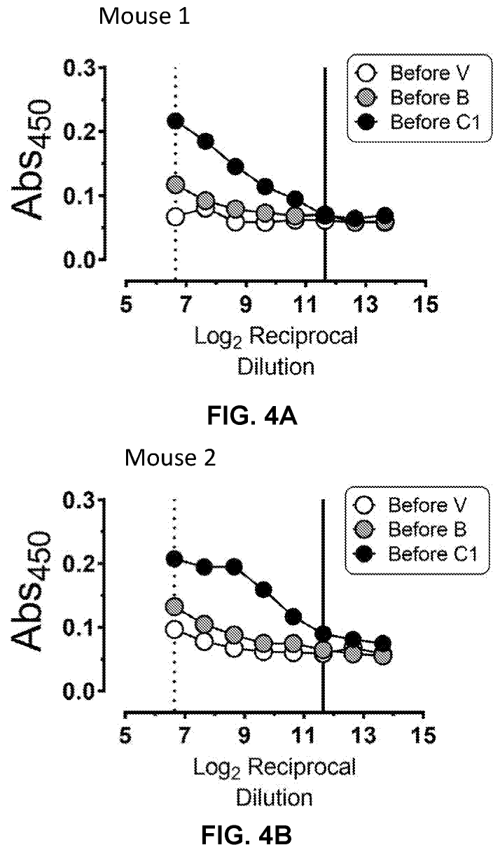

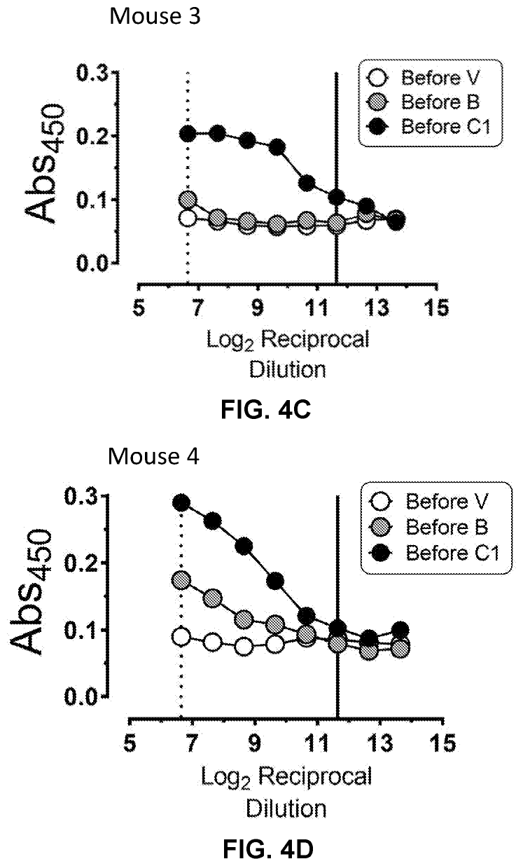

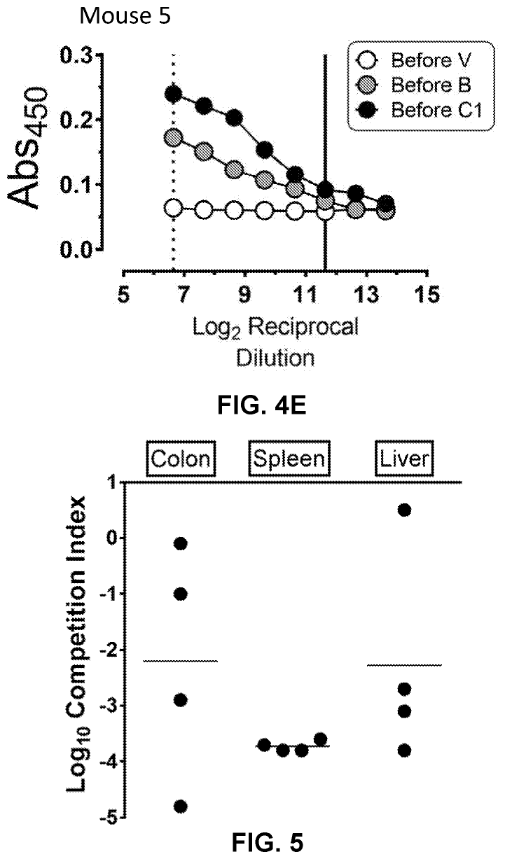

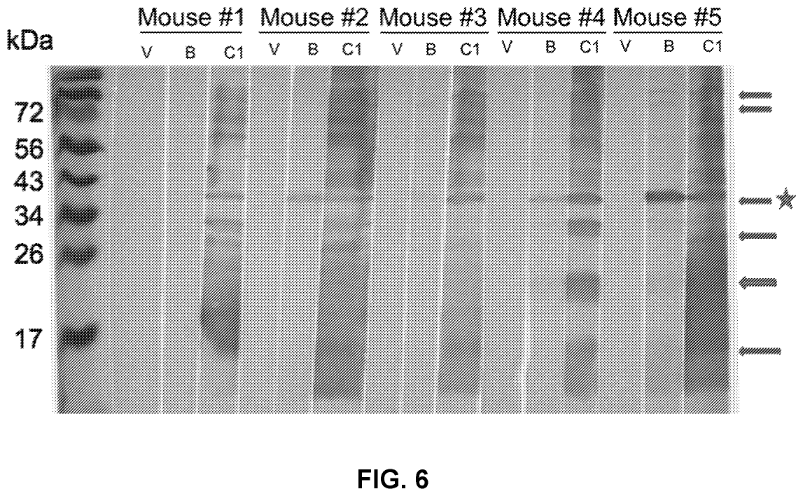

To measure the IgG response to NC983, serum was obtained through tail bleeding at one day prior to vaccination, boosting, and the 1^{st} challenge (FIG. 1). The mean endpoint titer taken before the boosting dose (13 dpv, before B) was 1:100 (dotted line, FDR adjusted p value=0.031; FIGS. 4A-4E). The mean endpoint titer after vaccination and boosting was 1:3,200 (solid line, FDR adjusted p value=0.018; FIGS. 4A-4E). Serum samples from individual mice were probed against whole cell lysates from S. Typhimurium by immunoblotting (FIG. 6) The data showed that serum taken before vaccination had no cross reactivity to S. Typhimurium antigens (lanes marked V). However, after vaccination 3 out of 5 mice showed a cross reactivity band at about 40 kDa (lanes marked B); but after the second inoculation (boosting) all mice showed multiple cross reactivity bands (lanes marked C1). Clearly, there was significant increase in cross reactivity to S. Typhimurium antigens from all mice at the C1 time point (i.e., just before the challenge) (FIG. 6). Indeed, further studies are needed to identify the different S. Typhimurium antigens reacting with the antibodies produced in the immunized mice.

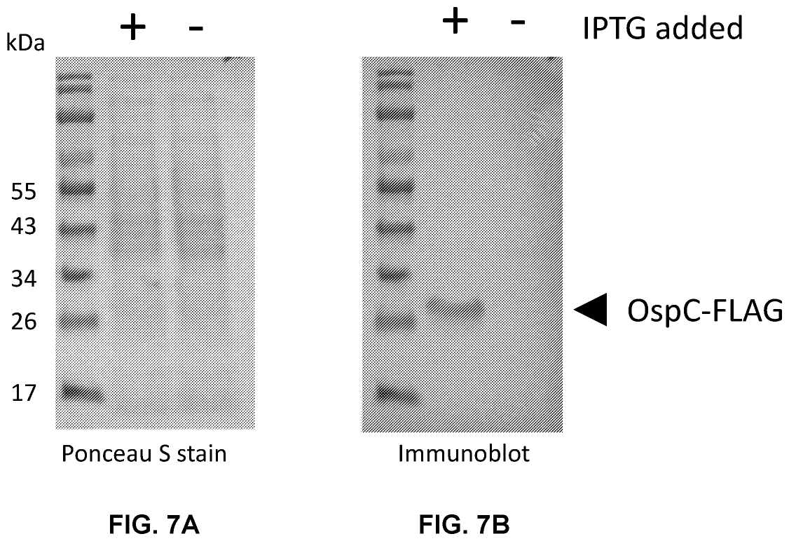

The expression of the heterologous antigen OspC from Borrelia burgdorferi in the vaccine strain (NC983, the modified S. Typhimurium strain) was examined. FIGS. 7A-7B show the ponceau S stain for protein (FIG. 7A) and corresponding immunoblot (FIG. 7B) demonstrating expression of the heterologous OspC antigen that was cloned into the modified S. Typhimurium strain. The sequenced expression vector plasmid containing the ospC-flag gene was cloned into the Typhimurium vaccine strain. IPTG was added to one culture for induction of the OspC-FLAG while the other culture did not contain IPTG. After four hours of induction, the cells were concentrated and treated for SDS-PAGE. Approximately, 10⁷ cells from each culture were loaded per lane. FIG. 7A is a Ponceua S stain of the membrane to demonstrate that equivalent levels of protein were present in each lane. FIG. 7B is the immunoblot that detected the OspC-FLAG protein of the expected size (about 26-27 kDa).

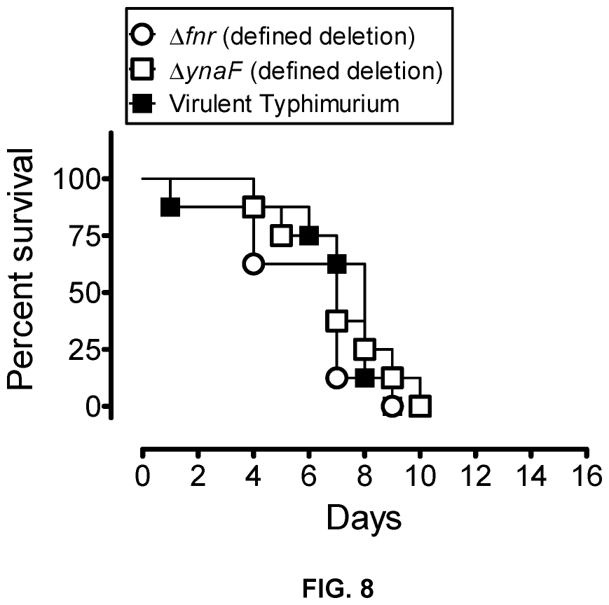

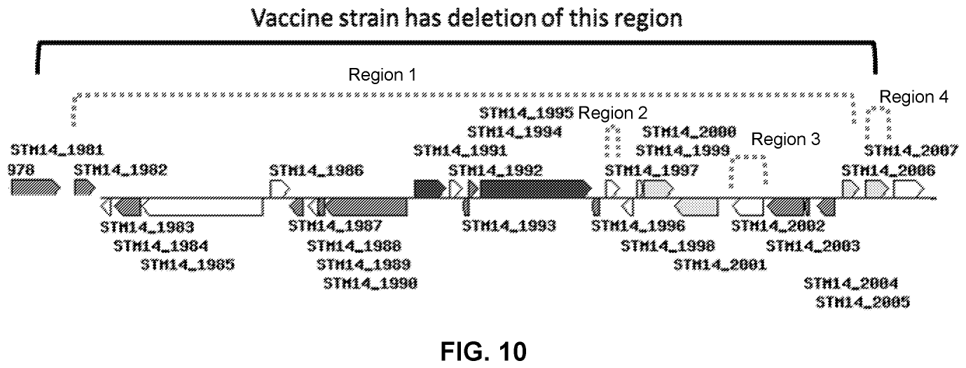

The NC983 (vaccine strain) contains a deletion of 25 genes and 2 truncated genes associated with attenuation virulence (solid bracket in FIG. 10). Different modified strains containing different gene deletions within the 25 genes that are deleted in the NC983 strain were generated and examined. FIG. 8 shows a graph demonstrating percent survival vs. time (days) post-infection of mice that were vaccinated with a strain that carried different defined deletions of one of two candidate genes (Δfnr or ΔynaF) or the parent (unmodified) virulent strain. Briefly, groups of four female C57BL/6 (6-10 weeks old) mice were challenged with the defined single mutants Δfnr, ΔynaF, or the virulent strain. Neither of these candidate genes did conferred the vaccine phenotype. In other words, neither of these two genes alone confer virulence phenotype, thus the lack of attenuation when either one was deleted.

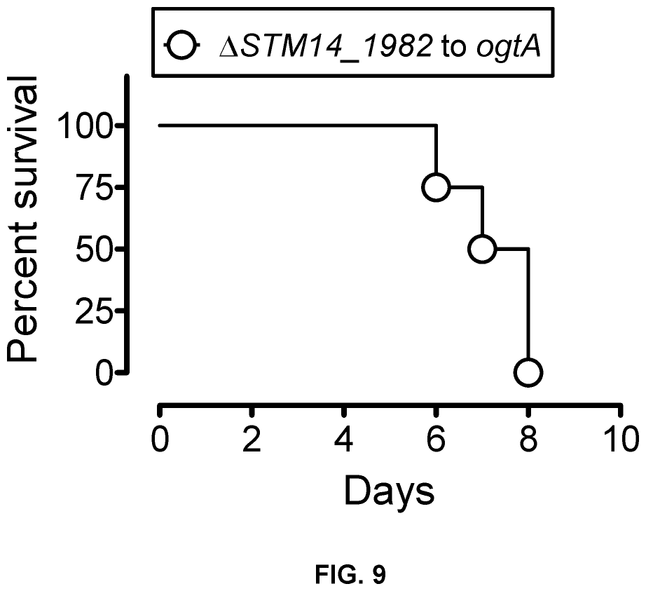

The NC983 (vaccine strain) contains a deletion of 25 genes and 2 truncated genes associated with the virulence phenotype (solid bracket in FIG. 10). A modified strain was made that contained defined deletions of 25 of the 27 candidate attenuation-associated genes that lie within the region of the NC983 strain. (Large dashed bracket in FIG. 10). Briefly, a group of four female C57BL/6 (6-10 weeks old) mice were challenged with the defined deletion that inactivated 25 of the 26 genes missing from the vaccine strain. As shown in FIG. 9, defined deletions of 25 of the 26 candidate genes within the missing region of the vaccine strain did not confer attenuation of virulence (vaccine phenotype).

FIG. 10 shows a cartoon summary of the genetic arrangements studies to examine the gene(s) involved in generating the vaccine (NC983) phenotype. The vaccine strain (NC983) has a deletion that inactivates at least 27 genes. As shown in of FIG. 10, 25 genes are completely removed and 2 are partially truncated. Systematic inactivation of two of the candidate genes responsible for the vaccine phenotype, fnr and ynaF, did not replicate the phenotype. In addition, inactivation of the 25 of the 27 genes did not replicate the vaccine phenotype. Genetic regions with a solid black bracket indicate a mutation that confers attenuation whereas a dashed bracket indicates that deletion of this region is still virulent. The largest dashed region (Region 1) corresponds to the deletion of 25 of the 27 genes that were deleted. The dashed region labeled Region 2 corresponds to the deletion of ynaF (STM14_1997) The dashed region labeled Region 3 corresponds to the deletion of zntB (STM14_2002) The dashed region labeled Region 4 corresponds to the deletion of fnr (STM 14_2007).

Summary.

Highly invasive S. Typhimurium and NTS isolates are becoming increasingly problematic in specific areas [28-31]. Therefore, there is a demand for effective preventative measures. This Example can at least demonstrate the effectiveness of a live attenuated S. Typhimurium strain (NC983) that fully protected two Salmonella sensitive mice strains from challenge with virulent S. Typhimurium. Strain NC983 was sporadically capable of reaching systemic tissues sites while exhibiting a pronounced fitness defect in the spleen. Collectively, these results support the at least that strain NC938 is attenuated and elicits protective immunity in mice models.

### References for Example 2

1. CDC: Surveillance for Foodborne Disease Outbreaks—United States, 2009-2010. MMWR Morb Mortal Wkly Rep 2013, 62(03):41-47.

2. Germanier R: Immunity in experimental salmonellosis. 3. Comparative immunization with viable and heat-inactivated cells of Salmonella typhimurium. Infect Immun 1972, 5(5):792-797.

3. Curtiss R 3^{rd}, Kelly S M, Hassan J O: Live oral avirulent Salmonella vaccines. Vet Microbiol 1993, 37(3-4):397-405.

4. Desin T S, Koster W, Potter A A: Salmonella vaccines in poultry: past, present and future. Expert Rev Vaccines 2013, 12(1):87-96.

5. Spreng S, Dietrich G, Weidinger G: Rational design of Salmonella-based vaccination strategies. Methods 2006, 38:133-143.

6. Hoiseth S K, Stocker B A: Aromatic-dependent Salmonella typhimurium are non-virulent and effective as live vaccines. Nature 1981, 291(5812):238-239.

7. Smith B P, Reina-Guerra M, Hoiseth S K, Stocker B A, Habasha F, Johnson E, Merritt F: Aromatic-dependent Salmonella typhimurium as modified live vaccines for calves. Am J Vet Res 1984, 45(1):59-66.

8. Smith B P, Reina-Guerra M, Stocker B A, Hoiseth S K, Johnson E: Aromatic-dependent Salmonella dublin as a parenteral modified live vaccine for calves. Am J Vet Res 1984, 45(11):2231-2235.

9. Mukkur T K, McDowell G H, Stocker B A, Lascelles A K: Protection against experimental salmonellosis in mice and sheep by immunisation with aromatic-dependent Salmonella typhimurium. J Med Microbiol 1987, 24(1):11-19.

10. Nnalue N A, Stocker B A: Test of the virulence and live-vaccine efficacy of auxotrophic and galE derivatives of Salmonella choleraesuis. Infect Immun 1987, 55(4):955-962.

11. Edwards M F, Stocker B A: Construction of delta aroA his delta pur strains of Salmonella typhi. J Bacteriol 1988, 170(9):3991-3995.

12. Levine M M, Herrington D, Murphy J R, Morris J G, Losonsky G, Tall B, Lindberg A A, Svenson S, Baqar S, Edwards M F et al: Safety, infectivity, immunogenicity, and in vivo stability of two attenuated auxotrophic mutant strains of Salmonella typhi, 541Ty and 543Ty, as live oral vaccines in humans. J Clin Invest 1987, 79(3):888-902.

13. Stocker B A: Aromatic-dependent salmonella as anti-bacterial vaccines and as presenters of heterologous antigens or of DNA encoding them. J Biotechnol 2000, 83(1-2):45-50.

14. Feigner S, Frahm M, Kocijancic D, Rohde M, Eckweiler D, Bielecka A, Bueno E, Cava F, Abraham W-R, Curtiss R3^{rd}, et al: aroA-deficient Salmonella enterica Serovar Typhimurium is more than a metabolically attenuated mutant. mBio 2016, 7(5):e01220-16.

15. Erova T E, Kirtley M L, Fitts E C, Ponnusamy D, Baze W B, Andersson J A, Cong T, Tiner B L, Sha J, Chpra A K: Protective immunity elicited by oral immunization of mice with Salmonella enterica Serovar Typhimurium Braun lipoprotein (Lpp) and acetyltransferase (MsbB) mutants. Front Cell Infect Microbiol 6:148.

16. de la Cruz M L, Conrado I, Nault A, Perez A, Dominguez L, Alvarez J: Vaccination as a control strategy against Salmonella infection in pigs: a systematic review and meta-analysis of the literature. Res Vet Sci 2017, 114:86-94.

17. Fink R C, Evans M R, Porwollik S, Vazquez-Torres A, Jones-Carson J, Troxell B, Libby S J, McClelland M, Hassan H M: FNR is a global regulator of virulence and anaerobic metabolism in Salmonella enterica serovar Typhimurium (ATCC 14028s). Journal of bacteriology 2007, 189(6):2262-2273.

18. Porwollik S, Santiviago C A, Cheng P, Long F, Desai P, Fredlund J, Srikumar S, Silva C A, Chu W, Chen X et al: Defined single-gene and multi-gene deletion mutant collections in Salmonella enterica sv Typhimurium. PLoS One 2014, 9(7):e99820.

19. Jarvik T, Smillie C, Groisman E A, Ochman H: Short-term signatures of evolutionary change in the Salmonella enterica serovar typhimurium 14028 genome. J Bacteriol 2010, 192(2):560-567.

20. Troxell B, Fink R C, Dickey A N, Scholl E H, Hassan H M: Complete Genome Sequence of NC983, a Live Attenuated Strain of Salmonella enterica Serovar Typhimurium. Genome Announc 2016, 4(5).

21. Troxell B, Petri N, Daron C, Pereira R, Mendoza M, Hassan H M, Koci M D: Poultry body temperature contributes to invasion control through reduced expression of Salmonella pathogenicity island 1 genes in Salmonella enterica serovars Typhimurium and Enteritidis. Appl Environ Microbiol 2015, 81(23):8192-8201.

22. Reed L Ja M H.: A simple method of estimating fifty percent endpoints. The American Journal of Hygiene 1938, 27:493-497.

23. Finney D J: Probit analysis: a statistical treatment of the sigmoid response curve. Cambridge, England: Cambridge University Press; 1952.

24. Freter R, O'Brien P C, Macsai M S: Role of chemotaxis in the association of motile bacteria with intestinal mucosa: in vivo studies. Infect Immun 1981, 34(1):234-240.

25. Ullman-Cullere M H, Foltz C J: Body condition scoring: a rapid and accurate method for assessing health status in mice. Lab Anim Sci 1999, 49(3):319-323.

26. Troxell B, Ye M, Yang Y, Carrasco S E, Lou Y, Yang X F: Manganese and zinc regulate virulence determinants in Borrelia burgdorferi. Infect Immun 2013, 81(8):2743-2752.

27. Troxell B, Xu H, Yang X F: Borrelia burgdorferi, a pathogen that lacks iron, encodes manganese-dependent superoxide dismutase essential for resistance to streptonigrin. J Biol Chem 2012, 287(23):19284-19293.

28. Graham S M: Nontyphoidal salmonellosis in Africa. Curr Opin Infect Dis 2010, 23(5):409-414.

29. Gordon MA: Invasive nontyphoidal Salmonella disease: epidemiology, pathogenesis and diagnosis. Curr Opin Infect Dis 2011, 24(5):484-489.

30. Ao T T, Feasey N A, Gordon M A, Keddy K H, Angulo F J, Crump J A: Global burden of invasive nontyphoidal Salmonella disease, 2010(1). Emerg Infect Dis 2015, 21(6).

31. Feasey N A, Dougan G, Kingsley R A, Heyderman R S, Gordon M A: Invasive non-typhoidal salmonella disease: an emerging and neglected tropical disease in Africa. Lancet 2012, 379(9835):2489-2499.

1.-2. (canceled)

3. An engineered Salmonella enterica serovar Typhimurium (S. Typhimurium) bacterium comprising:
a combination of gene deletions, where each gene in the combination of gene deletions are each independently selected from the group consisting of:
STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997, STM14_1998, STM14_1999, STM14_2000, STM14_2001, STM14_2002, STM14_2003, STM14_2004, STM14_2005, STM14_2006, and STM14_2007, wherein the combination of gene deletions is not the combination of STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997 STM14_1998, STM14_1999, STM14_2000, STM14_2001, STM14_2002, STM14_2003, STM14_2004, STM14_2005, and STM14_2006.

4. The engineered S. Typhimurium bacterium of claim 3, wherein the engineered S. Typhimurium bacterium has decreased virulence as compared to wild-type S. Typhimurium.

5. The engineered Salmonella enterica serovar Typhimurium (S. Typhimurium) bacterium of claim 3, wherein the combination of gene deletions consists of 26 deleted genes and wherein the 26 deleted genes are STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997, STM14_1998, STM14_1999, STM14_2000, STM14_2001, STM14_2002, STM14_2003, STM14_2004, STM14_2005, and STM14_2006.

6. The engineered S. Typhimurium bacterium of claim 5, wherein the engineered S. Typhimurium bacterium has decreased virulence as compared to wild-type S. Typhimurium.

7. The engineered S .Typhimurium bacterium of claim 3, wherein the combination of gene deletions consists of 24 deleted genes and wherein the 24 deleted genes are STM14 1981, STM14 1982, STM14 1983, STM14 1984, STM14 1985, STM14 1986, STM14 1987, STM14 1988, STM14 1989, STM14 1990, STM14 1991, STM14 1992, STM14 1993, STM14 1994, STM14 1995, STM14 1996, STM14 1998, STM14 1999, STM14 2000, STM14 2001, STM14 2003, STM14 2004, STM14 2005, and STM14 2006.

8. The engineered S. Typhimurium bacterium of claim 7, wherein the engineered S. Typhimurium bacterium has decreased virulence as compared to wild-type S. Typhimurium.

9. The engineered S. Typhimurium bacterium of claim 3, wherein the the engineered S. Typhimurium further comprises a foreign epitope.

10-19. (canceled)

20. A vaccine comprising:
an engineered Salmonella enterica serovar Typhimurium (S. Typhimurium) bacterium comprising:
a combination of gene deletions, where each gene in the combination of gene deletions are each independently selected from the group consisting of: STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997, STM14_1998, STM14_1999, STM14_2000, STM14_2001, STM14_2002, STM14_2003, STM14_2004, STM14_2005, STM14_2006, and STM14_2007, wherein the combination of gene deletions is not the combination of STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997 STM14_1998, STM14_1999, STM14_2000, STM14_2001 STM14_2002 STM14_2003 STM14_2004 STM14_2005 and STM14_2006.

21. The vaccine of claim 20, wherein the combination of gene deletions consists of 26 deleted genes and wherein the 26 deleted genes are STM14_1981, STM14_1982, STM14_1983 STM14_1984, STM14_1985, STM14_1986, STM14_1987, STM14_1988, STM14_1989 STM14_1990, STM14_1991, STM14_1992, STM14_1993, ,STM14_1994, STM14_1995 STM14_1996, STM14_1997, STM14_1998, STM14_1999, STM14_2000, STM14_2001 STM14_2002 STM14_2003 STM14_2004 STM14_2005 and STM142006.

21. The vaccine of claim 20, wherein the combination of gene deletions consists of 24 deleted genes and wherein the 24 deleted genes are STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1998, STM14_1999, STM14_2000, STM14_2001, STM14_2003, STM14_2004, STM14_2005, and STM14_2006.

22. The vaccine of claim 20, wherein the engineered S. Typhimurium_further comprises a foreign epitope.

23. The vaccine of claim 20, further comprising an adjuvant.

24. The vaccine of claim 20, wherein the vaccine contains from about 10⁷ colony forming units of the engineered S. Typhimurium to about 10⁸ colony forming units of the engineered S. Typhimurium.

25. A method comprising:
administering a vaccine to a subject in need thereof, wherein the vaccine comprises an engineered Salmonella enterica serovar Typhimurium (S. Typhimurium) bacterium comprising:
a combination of gene deletions, where each gene in the combination of gene deletions are each independently selected from the group consisting of: STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997, STM14_1998, STM14_1999, STM14_2000, STM14_2001, STM14_2002, STM14_2003, STM14_2004, STM14_2005, STM14_2006, and STM14_2007, wherein the combination of gene deletions is not the combination of STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997 STM14_1998, STM14_1999, STM14_2000, STM14_2001, STM14_2002, STM14_2003, STM14_2004, STM14_2005, and STM14_2006.

26. The method of claim 25, wherein the combination of gene deletions consists of 26 deleted genes, wherein the 26 deleted genes are STM14_1981, STM14_1982, STM14_1983, STM14_1984, STM14_1985, STM14_1986, STM14_1987, STM14_1988, STM14_1989, STM14_1990, STM14_1991, STM14_1992, STM14_1993, STM14_1994, STM14_1995, STM14_1996, STM14_1997, STM14_1998, STM14_1999, STM14_2000, STM14_2001, STM14_2002, STM14_2003, STM14_2004, STM14_2005, and STM14_2006.

27. The method of claim 25, wherein the combination of gene deletions consists of 24 deleted genes and wherein the 24 deleted genes are STM14_1981, STM14_1982, STM14 1983 STM14_1984, STM14_1985, STM14_1986, STM14_1987, STM14_1988, STM14 1989 STM14_1990, STM14_1991, STM14_1992, STM14_1993, STM14_1994, STM14 1995 STM14_1996, STM14_1998, STM14_1999, STM14_2000, STM14_2001, STM14_2003 STM14_2004 STM14_2005 and STM14_2006.

28. The method of claim 25, wherein the engineered S. Typhimurium_further comprises a foreign epitope.

29. The method of claim 25, wherein the vaccine contains from about 10⁷ colony forming units of the engineered S. Typhimurium to about 10⁸ colony forming units of the engineered S. Typhimurium.

30. The method of claim 25, wherein the vaccine is effective to induce an immune response in the subject.

31. The method of claim 25, wherein the vaccine is effective to treat or prevent Salmonella enterica serovar Typhimurium infection in the subject in need thereof.
